(19) United States
(12) Patent Application Publication
(10) Pub. No.: US 2008/0091086 A1
(43) Pub. Date: Apr. 17, 2008

(54) Method and Apparatus for a Constellation-of-Symptoms Approach to Patient-Driven Computer-Assisted Diagnosis

(76) Inventors: Henry Joseph Legere, Malden, MA (US); Nicholas Aaron Solter, Colorado Springs, CO (US)

Correspondence Address: NICHOLAS SOLTER, 13010 ROCKBRIDGE CIRCLE, COLORADO SPRINGS, CO, 80921, US

(21) Appl. No.: 11/869,377

(22) Filed: Oct. 9, 2007

Related U.S. Application Data
(60) Provisional application No. 60/829,253, filed on Oct. 12, 2006.

(51) Int. Cl. A61B 5/00

(52) U.S. Cl. 600/300

(57) ABSTRACT

One embodiment of the present invention provides a computer software medical diagnosis system with which users can interact over the Internet using a web browser in order to obtain an ordered list of possible diagnoses for their current constellation of symptoms. The user interacts with the diagnosis system by specifying a list of current symptoms. Then the diagnosis system presents him or her with a list of possible diagnoses for those symptoms ordered according to their likelihood of explaining the constellation of symptoms.

This application claims the benefit of U.S. Provisional Application No. 60/829,253 filed Oct. 12, 2006.

### FIELD OF THE INVENTION

The present invention relates to the field of computer-assisted medical diagnosis, and, more specifically, to a constellation-of-symptoms approach to patient-driven computer-aided diagnosis.

### RELATED ART

The diagnosis of a patient's illness is a complex activity in which a physician or medical professional aggregates information from the patient's statements and answers to questions, the patient's health history, physical findings, lab results, and other sources. The physician uses his or her expertise and medical training to reach a conclusion about the source of the patient's ailments.

The idea of computer-assisted diagnosis has become popular for several reasons, among them the scarcity of physicians, the perceived mistakes of medical professionals, the growing ubiquity of computers, and the perceived infallibility of computers. The “holy grail” of computer-assisted diagnosis is a system with which a user can interact with the computer to obtain a diagnosis without the presence or assistance of a medical professional.

There has been much diverse research in the field of computer-assisted diagnosis. The traditional “expert system” approach to computer-assisted diagnostic systems is to assemble a database of diseases and findings and to connect them with probabilities. An example of this approach is the Internist-1 system (Miller, R. A., Pople, H. E., Jr., Myers, J. D.: Internist I, An Experimental Computer-Based Diagnostic Consultant for General Internal Medicine. The New England Journal of Medicine, 307:468-476, 1982).

Another approach is to formalize physicians' logic in the form of diagnosis trees, or “diagnosis algorithms.” The computer system can then run these algorithms for the user. An example of this approach is disclosed in patent application number 11869292, “Method and Apparatus for an Algorithmic Approach to Patient-Driven Computer-Assisted Diagnosis,” by Dr. Henry Joseph Legere and Nicholas Aaron Solter.

Even though the algorithmic approach to computer-assisted diagnosis better models a physician's thinking than does the probabilistic approach, it still hides some aspects of the physician decision-making process. When physicians diagnose patients they typically start with a “differential diagnosis” of all possible diseases given the patient's collection, or “constellation,” of symptoms. They then ask further questions, perform physical exams, or order lab tests in order to narrow down the diagnosis.

When interacting with a computer, instead of a live physician, some patients may not trust a categorical diagnosis, or may not want to spend the time to answer a multitude of questions in order to arrive at a diagnosis. Instead patients may prefer to use the computer for a quick symptom check to figure out the possible diseases that could be causing their symptoms. Essentially, they would like to be able to access the differential diagnosis for their constellation of symptoms.

### SUMMARY OF THE INVENTION

One embodiment of the present invention provides a computer software medical diagnosis system with which users can interact over the Internet using a web browser in order to obtain an ordered list of possible diagnoses for their current constellation of symptoms. The user interacts with the diagnosis system by specifying a list of current symptoms. Then the diagnosis system presents him or her with a list of possible diagnoses for those symptoms ordered according to their likelihood of explaining the constellation of symptoms.

In this embodiment of the invention, the medical information used by the diagnosis system takes the form of a list of possible symptoms, a list of possible diagnoses, and mappings of symptom constellations to an ordered list of diagnosis codes.

In this embodiment, each constellation of symptoms has a unique integer id, generated from the unique ids for each individual symptom. The diagnoses, symptoms, and mappings of symptom constellations to diagnosis codes are all stored in-memory in hashtables.

In this embodiment, the diagnosis system begins a user interaction by presenting a list of symptoms to the user. The system retrieves the user's symptom selection, generates the unique id for that symptom constellation, retrieves the ordered diagnosis code list for that constellation of symptoms, uses the diagnosis code list to lookup each diagnosis, and finally displays the ordered list of diagnoses to the user.

In a variation on this embodiment, each constellation of symptoms is associated with two or more separate ordered lists of diagnoses, in order to distinguish between common and rare etiologies.

### DESCRIPTION OF DRAWINGS

Exemplary embodiments of the invention will be described with reference to the accompanying drawings. Like items in the drawings are shown with the same reference numbers. Embodiments of the invention are illustrated by way of example, and not by way of limitation, in the figures of the accompanying drawings.

FIG. 1 is a block diagram depicting the computer-assisted diagnosis according to an embodiment.

FIG. 2 is a flow diagram depicting the computer-assisted diagnosis according to an embodiment.

FIG. 3 is list of symptoms in accordance with an embodiment.

FIG. 4 is a list of diagnosis codes and diagnoses in accordance with an embodiment.

FIG. 5 is a list of mappings from symptom constellations to diagnosis codes, in accordance with an embodiment.

FIG. 6 is a block diagram depicting a computer system implementing the computer-assisted diagnosis according to an embodiment.

FIG. 7 is a block diagram of the in-memory representation of the medical data in accordance with an embodiment.

FIG. 8 is a flow diagram depicting the operation of the diagnosis system in accordance with an embodiment.

### DETAILED DESCRIPTION OF THE INVENTION

In the following description, numerous specific details are set forth in order to provide a thorough understanding of the present invention and to enable any person skilled in the art to make and use the invention. In some instances, well-known features have not been described in detail to avoid unnecessarily obscuring the present invention. Various modifications to the disclosed embodiments will be readily apparent to those skilled in the art, and the general principles defined herein may be applied to other embodiments and applications without departing from the spirit and scope of the present invention. Thus, the present invention is not intended to be limited to the embodiments shown, but is to be accorded the widest scope consistent with the principles and features disclosed herein.

Various aspects and features of example embodiments of the invention are described in more detail hereinafter in the following sections: (1) Functional Overview; (2) Medical Information; (3) Implementation; (4) Variations.

### Functional Overview

One or more embodiments of the invention relate to a computer software medical diagnosis system with which users can interact to obtain an ordered list of possible diagnoses for their “constellation of symptoms.” As used herein, the term, “Constellation of Symptoms” refers to the set of related symptoms that the user is currently experiencing. The diagnosis system works by querying the user for a constellation of symptoms and then presenting a list of possible diagnoses for those symptoms ordered according to their likelihood of being the cause of those symptoms.

In one or more embodiments of the invention, the interaction between the user and the diagnosis program emulates a physician-patient relationship, but does not involve a live physician. Instead, the diagnosis software relies on a database of medical information. However, those skilled in the art will appreciate that the user could in fact be a medical professional operating the diagnosis program in the process of diagnosing a patient.

In one or more embodiments of the invention, the user interacts with the diagnosis system over the Internet. The diagnosis system itself runs on a server attached to the Internet, and the user interacts with the server over the World Wide Web through his or her web browser. Those skilled in the art will appreciate that this interaction may take other forms, including a user interacting with the diagnosis system through input/output devices connected directly to the diagnosis system, a user interacting with the diagnosis system on a hand-held computer, and the like. The invention is not limited to a particular medium of interaction.

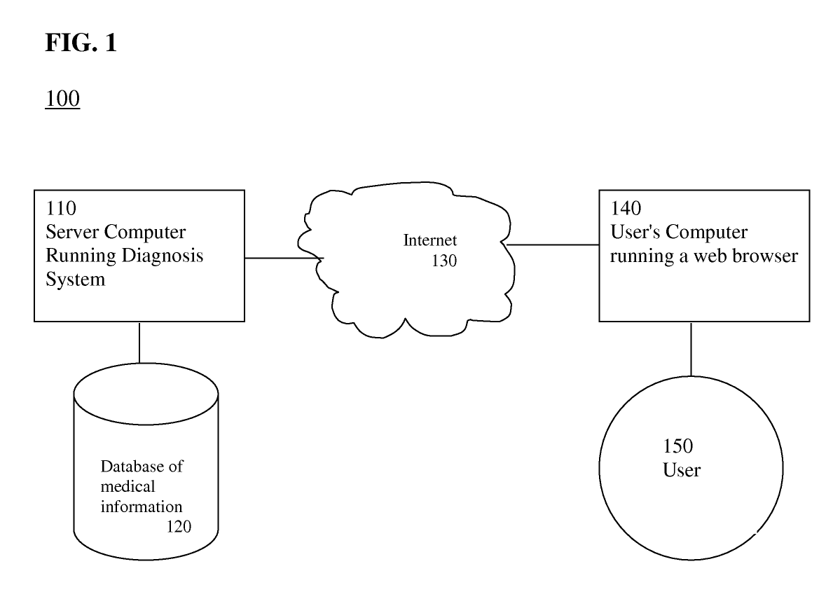

FIG. 1 is a block diagram, 100, of a user interacting with the diagnosis software in accordance with one or more embodiments of the present invention. A user, 150, accesses the diagnosis functionality through a web browser running on his or her computer, 140. The user's web browser contacts the diagnosis program running on the server computer, 110, through the Internet, 130. The server computer accesses a database of medical information, 120.

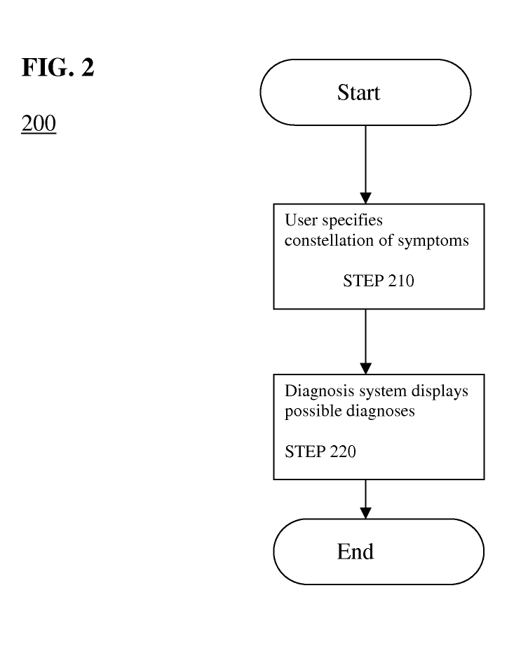

The diagnosis program just described is now described with reference to a flow diagram 200 of FIG. 2. A user specifies his or her constellation of symptoms in STEP 210. The diagnosis system displays an ordered list of possible diagnoses for those symptoms in STEP 220.

### Medical Information

In one or more embodiments of the invention, the medical information used by the diagnosis system takes the form of a list of possible symptoms, a list of possible diagnoses, and mappings of symptom constellations to diagnosis codes. The diagnoses are each assigned a unique numerical code for easy reference. The numbers may be standard ICD9 codes, MeSH codes, proprietary codes, or the like. The invention is not limited to any type of diagnosis code. The list of diagnosis codes for each symptom constellation is ordered according to the likelihood of the disease explaining the present symptoms.

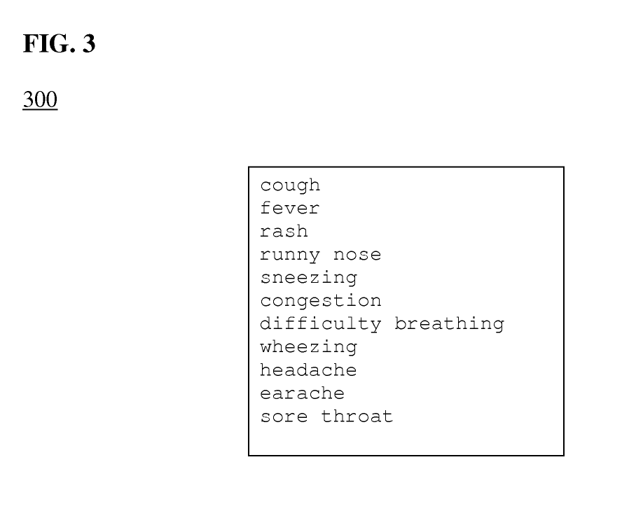

FIG. 3 shows a list of respiratory symptoms in accordance with an embodiment.

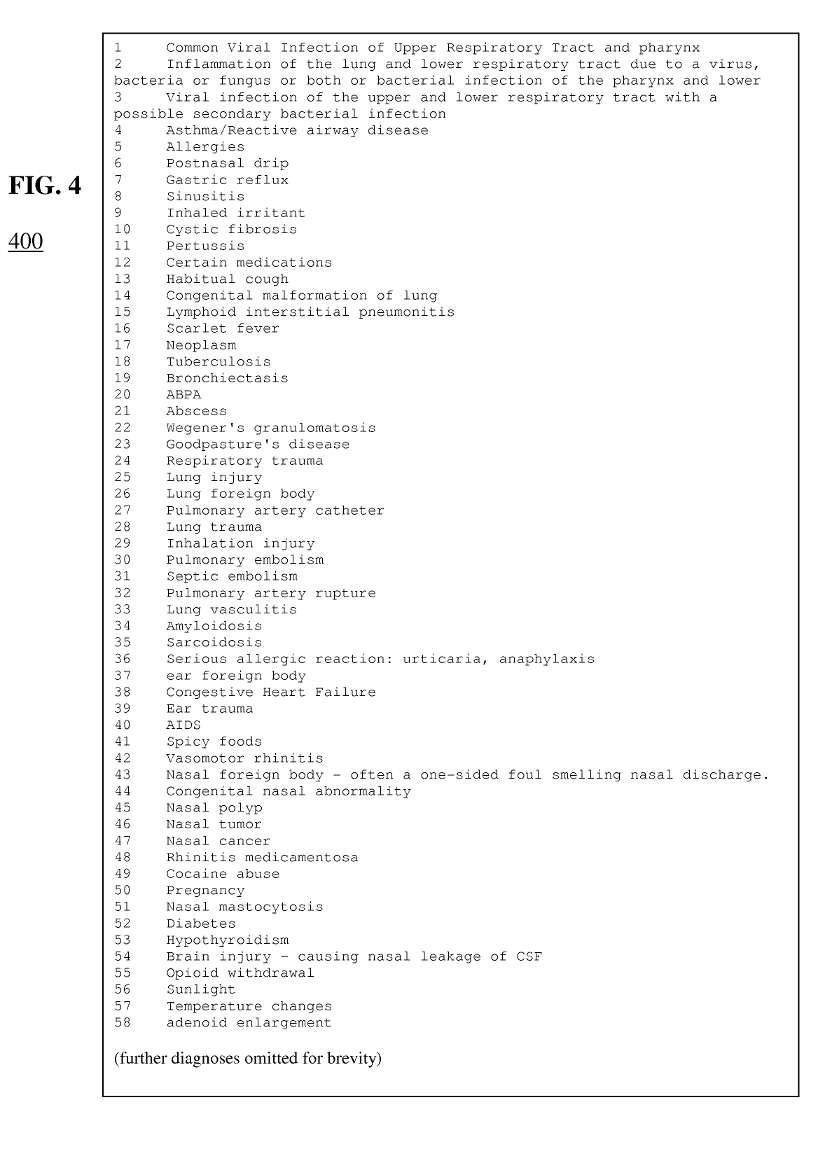

FIG. 4 shows a list of respiratory system-related diagnosis codes and diagnoses in accordance with an embodiment.

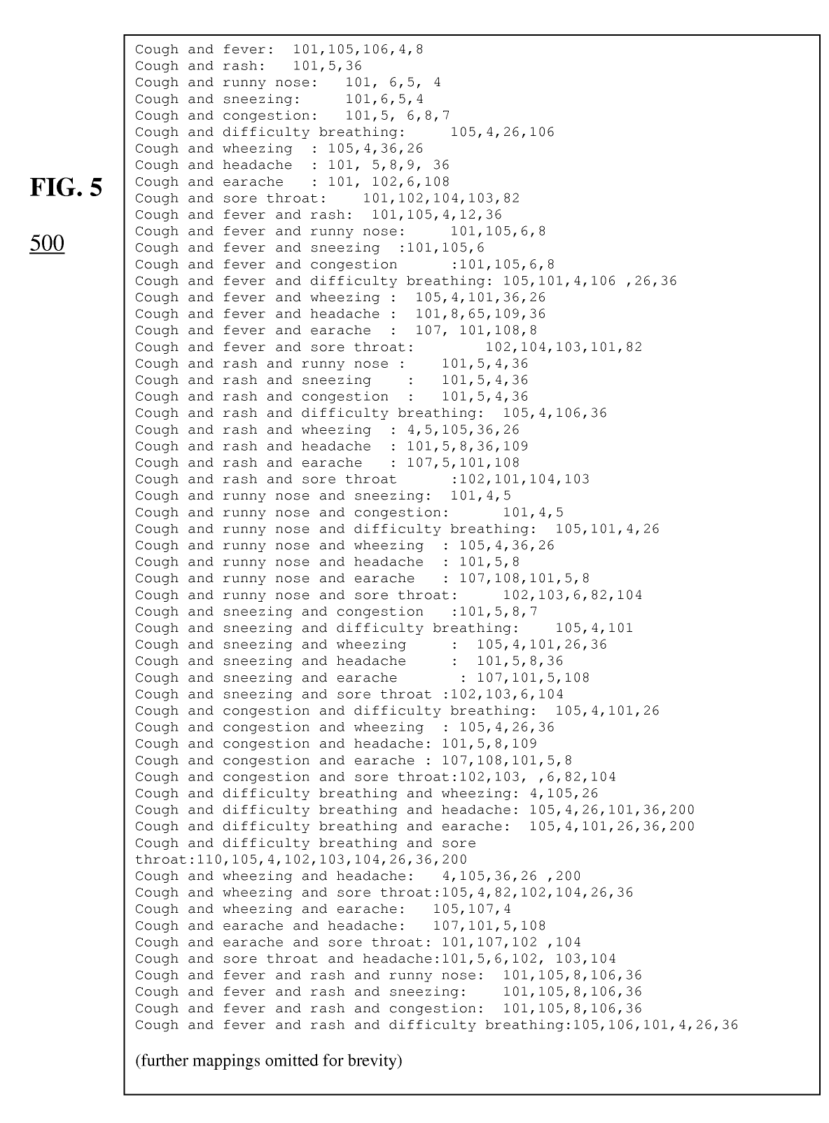

FIG. 5 shows a list of mappings from symptom constellations to diagnosis codes, in accordance with an embodiment.

### Implementation

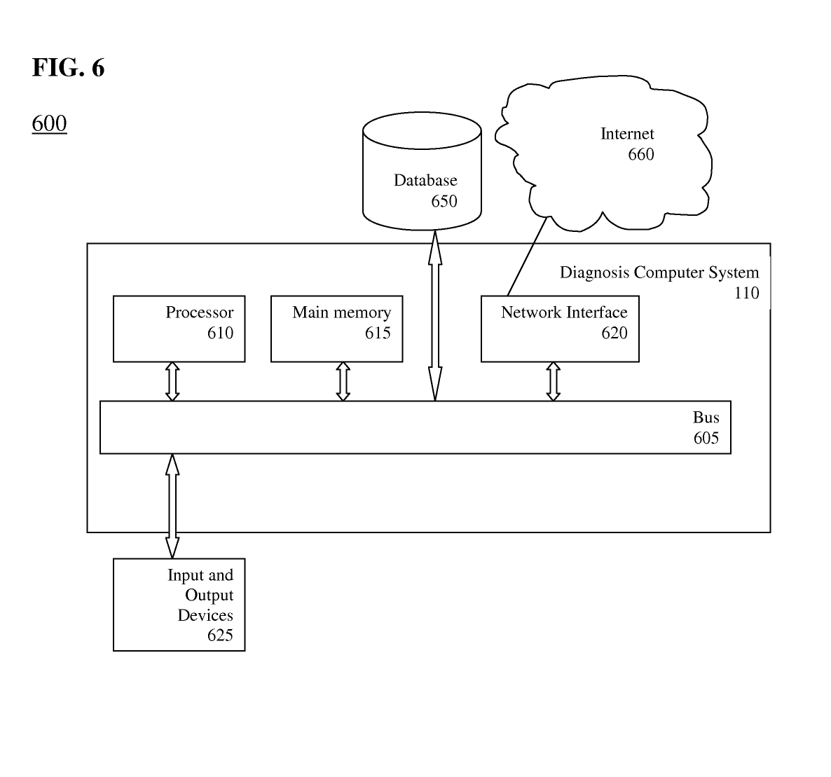

In one or more embodiments, the diagnosis system may be implemented on virtually any type of computer regardless of the platform being used. FIG. 6 shows a computer system 110 that implements the diagnosis software in accordance with one or more embodiments of the invention. This computer system includes a processor 610, associated memory 615, a bus or other communication mechanism 605, a network interface 620, a database 650, and numerous other elements and functionalities typical of today's computers (not all of which are shown).

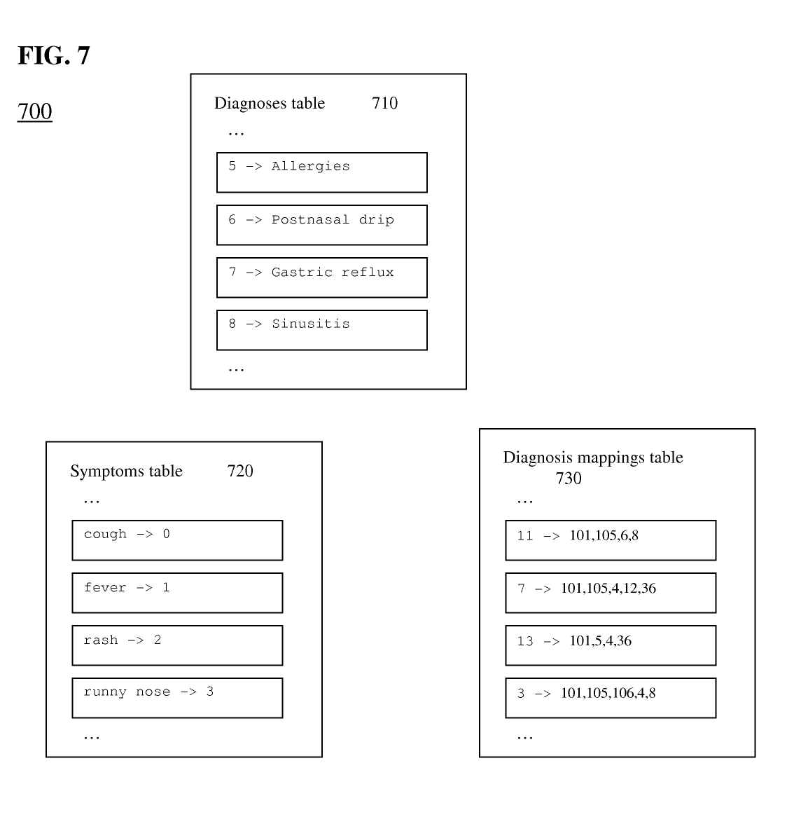

FIG. 7 is a block diagram of the in-memory representation of the medical data in accordance with one or more embodiments. This representation is explained with reference to FIG. 7.

In one or more embodiments, the diagnoses are stored in-memory in a hashtable, keyed on the diagnosis code (710 in FIG. 7).

In one or more embodiments, the diagnosis symptoms are each assigned a unique integer id starting from 0. The symptoms and associated ids are stored in-memory in a hashtable, keyed on the symptom name (720 in FIG. 7).

In one or more embodiments, unique symptom constellation ids are represented as bitmaps. The id for each constellation is obtained by setting to 1 the bits represented by the symptom ids for all symptoms that make up the constellation and to 0 all other bits. With reference to FIG. 7, the symptom id for cough is 0, fever is 1, rash is 2, and runny nose is 3. The id for the constellation of cough, fever, and runny nose would set bits 0, 1, and 3 to 1 and all other bits to 0. With the least-significant-bit on the right, this bitmap is 1011.

In one or more embodiments, the mappings of symptom constellation to diagnosis codes are stored in-memory in a hashtable keyed on the decimal representation of the symptom constellation unique id (730 in FIG. 7). The value of each mapping is an ordered list of diagnoses for that symptom constellation. For example, assuming the symptom ids from 720 in FIG. 7, the id for the constellation of cough, fever, and runny nose is 1011 in binary, or 111 in decimal. The value of the mapping is “101,105,6,8,” which means that the diagnosis with code 101 is the most likely diagnosis for that symptom constellation, the diagnosis with code 105 is the second-most likely, etc. The first entry in table 730 in FIG. 7 shows this example. As a second example, the id for the constellation of cough, fever, and rash would be 0111 in binary, which is 7 in decimal. In 730 in FIG. 7 it maps to a diagnosis list of “101,105,4,12,36.” In a variation on this embodiment, if the number of symptoms exceeds the number of bits in the computer's representation of integers, a trie can be used to store the diagnoses mappings instead of a hashtable.

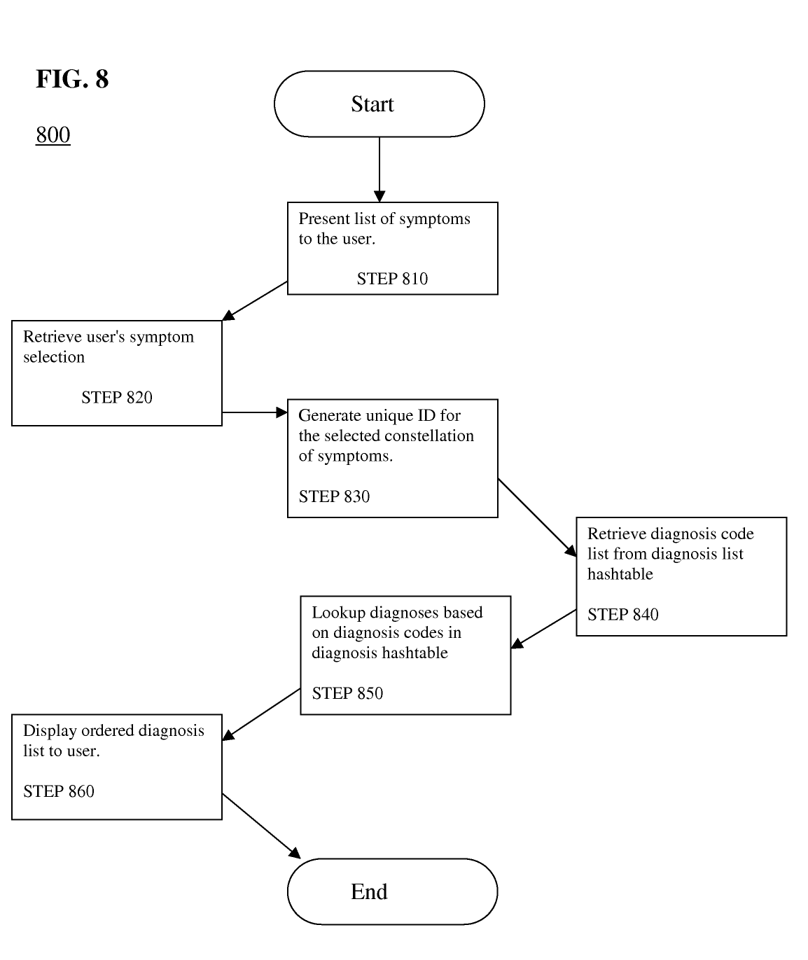

FIG. 8 shows a flow chart for the execution of the diagnosis system in accordance with one or more embodiments of the invention. The steps of the diagnosis system are described with reference to FIG. 8. In STEP 810, the diagnosis system begins a user interaction by presenting a list of symptoms to the user. The system retrieves the user's symptom selection in STEP 820. In STEP 830, the diagnosis system generates the unique id for that symptom constellation. The diagnosis system, in STEP 840, then uses the decimal representation of that key to retrieve the ordered diagnosis code list for that constellation of symptoms from the diagnosis list hashtable. In STEP 850, the diagnosis system uses the diagnosis code list to lookup each diagnosis in the diagnosis hashtable. Finally, in STEP 860, the diagnosis system displays the ordered list of diagnoses to the user.

### Variations

In one or more embodiments, each constellation of symptoms is associated with two or more separate ordered lists of diagnoses, in order to distinguish between common and rare etiologies.

What is claimed is:

1. A method for computer-assisted diagnosis, the method comprising:
encoding medical diagnosis information as a list of possible symptoms, a list of possible diagnoses, and mappings of combinations of symptoms (“symptom constellations”) to an ordered list of diagnoses;
a user initiating the diagnosis by specifying a symptom constellation; and
the user receiving an ordered list of possible diagnoses for that constellation of symptoms.

2. The method of claim 1, further comprising the lack of interaction, either directly or indirectly, of the user with a live physician.

3. The method of claim 1, further comprising assigning unique numerical diagnosis codes to each diagnosis.

4. The method of claim 3, wherein the mappings of symptom constellations to diagnoses are comprised of a list of diagnosis codes ordered according to the likelihood of the diagnosis represented by that code explaining the symptoms.

5. The method of claim 1, further comprising mappings of symptom constellations to two or more ordered lists of diagnoses.

6. The method of claim 4, wherein the diagnosis system presents a list of symptoms to the user, retrieves the user's symptom constellation selection, retrieves the diagnosis codes for that symptom constellation from the in-memory mapping of symptom constellations to diagnosis codes, retrieves each diagnosis from the in-memory mapping of diagnosis codes to diagnoses, and displays the ordered list of diagnoses to the user.

7. An apparatus for computer-assisted diagnosis, the apparatus comprising:
medical diagnosis information encoded in computer-readable media as a list of possible symptoms, a list of possible diagnoses, and mappings of combinations of symptoms (“symptom constellations”) to an ordered list of diagnoses;
an interface for a user to initiate a diagnosis by specifying a symptom constellation; and
an interface for the user to receive an ordered list of possible diagnoses for that constellation of symptoms.

8. The apparatus of claim 7, wherein the user interacts with the system over the Internet by using a web browser.

9. The apparatus of claim 7, further comprising an in-memory mapping from unique numerical diagnosis codes to diagnoses.

10. The apparatus of claim 9, further comprising:
a unique integer ID (“symptom ID”), starting from 0, for each symptom; and
an in-memory mapping of symptom names to symptom IDs.

11. The apparatus of claim 10, further comprising a unique bitmap for each symptom constellation, generated by setting to 1 the bits represented by the symptom IDs for all symptoms that make up the constellation and to 0 all other bits.

12. The apparatus of claim 11, further comprising an in-memory mapping of the unique symptom constellation bitmaps to an ordered list of diagnosis codes.

13. The apparatus of claim 12, wherein the in-memory mapping is implemented as a trie.

14. The apparatus of claim 12, wherein the in-memory mapping is implemented as a hashtable.

15. The apparatus of claim 12, wherein the diagnosis system presents a list of symptoms to the user, retrieves the user's symptom constellation selection, generates the unique bitmap for that symptom constellation, retrieves the diagnosis codes for that symptom constellation from the in-memory mapping of symptom constellation bitmaps to diagnosis codes, retrieves each diagnosis from the in-memory mapping of diagnosis codes to diagnoses, and displays the ordered list of diagnoses to the user.

16. The apparatus of claim 7, further comprising mappings of symptom constellations to two or more ordered lists of diagnoses.
